(19) United States
(12) Patent Application Publication
(10) Pub. No.: US 2010/0057182 A1
(43) Pub. Date: Mar. 4, 2010

(54) Method and device for loading a stent applicator

(76) Inventors: Kevin Pilz, Tuttlingen (DE); Rainer Hermle, Gosheim (DE)

Correspondence Address: ST. ONGE STEWARD JOHNSTON & REENS, LLC, 986 BEDFORD STREET, STAMFORD, CT, 06905-5619, US

(21) Appl. No.: 12/534,218

(22) Filed: Aug. 3, 2009

(30) Foreign Application Priority Data
Aug. 2, 2008 (DE) 10 2008 036 205.0

(51) Int. Cl. A61F 2/84

(52) U.S. Cl. 623/1.11

(57) ABSTRACT

A method and a device for loading a stent applicator that makes possible the loading of the stent applicator with simple handling and without risk of injury. The method is characterized by the steps of Exerting a lateral pressure on the stent to reduce its diameter, Inserting the compressed stent into a loading device that includes a cylinder-piston arrangement, At least partially inserting the loading device into the stent applicator, and Loading the stent applicator with the stent by pushing the components—cylinder and piston—of the loading device relative to one another.

### CROSS-REFERENCE TO RELATED APPLICATIONS

The present application claims priority of German patent application No. 10 2008 036 205.0 filed on Aug. 2, 2008.

### FIELD OF THE INVENTION

The invention relates to a method and a device for loading a stent applicator.

### BACKGROUND OF THE INVENTION

A stent is understood to refer to a small tube inserted endoscopically during surgery for bridging or drainage in cases of stenoses, strictures, and tumors. Stents take the form of tube-shaped prostheses produced from various materials that are compatible with the human body and serve, after the endoscopic or radiological implantation or in short-term vessel stenoses caused by arteriosclerosis, to bridge or to contain the lumen of a hollow organ.

Patent DE 10249 927 B3, for instance, discloses a device for compressing tubular endoprostheses as well as for inserting a compressed endoprosthesis into an application tube. This known device serves to compress stents made of pliable elastic material by forming a longitudinal fold, and subsequently to move the stents compressed in such manner by means of an ejector tool into an application tube.

In addition to stents made of elastic and primarily a synthetic material, however, there are also wire stents, for the most part made of a wire mesh. These wire stents can be reduced in diameter by means of lateral pressure, but it is not possible, as a rule, to construct a longitudinal fold that can reversibly reconstruct itself because of the material rigidity. For this reason, in the art the wire and wire-mesh stents are inserted manually into the stent applicators. This manual loading is very burdensome because the stent tends to exceed the diameter of the applicator and thus on being pushed inward it can become jammed. In addition, on the ends of the wire-mesh stents there are usually sharp wires extending, on which the operator can easily become injured while inserting the stent into the stent applicator.

It is consequently the object of the invention to create a method and a device for loading a stent applicator which make possible the loading of a stent applicator under conditions of easy operation and without risk of injury.

### SUMMARY OF THE INVENTION

The fulfillment of this object, in terms of the method, is characterized by the following steps:
a) Exert a lateral pressure force on the stent to reduce its diameter.
b) Insert the compressed stent into a loading device that comprises a cylinder-piston arrangement.
c) At least partly insert the loading device into the stent applicator.
d) Load the stent applicator with the stent by pushing the cylinder and piston components of the loading device relative to one another.

With the inventive method it is also possible to move wire stents into a stent applicator without risk of injury to the operator. The mere exerting of a lateral pressure force on the stent to reduce its diameter in step a) of the method occurs manually in one variant of the method, but this manual actuation involves no risk of injury to the person executing this method, because the stent is compressed from the sides, but not over the ends of the stent that include pointed wire ends.

According to an alternative embodiment of the step a) of the method, it is proposed according to the invention that the lateral pressure is exerted on the stent by means of a pressing device.

The pressing device for exerting a lateral pressure on the stent to reduce its diameter, according to a practical embodiment of the invention, comprises at least one pressure means that surrounds the periphery of the stent at least partially and by which the lateral pressure is transmitted onto the stent.

According to a first embodiment for configuring the press device, it is proposed that the at least one pressure means should be configured as jaw members of a forceps-type tool.

According to a second embodiment for configuring the pressing device, it is proposed that the at least one pressing means should be configured as a flexible band that surrounds the stent and can be drawn together, such as a cable tie that is known per se.

The actual insertion of the stent with reduced diameter into the loading device is performed, according to the invention, by way of a lateral slit in the cylinder of the loading device. This type of insertion of the stent into the loading device is advantageous because it requires no exertion of pressure on the ends that bear the pointed wire ends.

To move the stent into the stent applicator, it is proposed according to a first embodiment of the invention that, in step d) of the method, to empty the loading device the piston should be held firmly and the cylinder should be moved away from the stent applicator. This manner of emptying the loading device has the advantage that the stent is not required to be pushed inside the applicator.

According to a second embodiment of the inventive method, it is proposed that in step d) of the method, to empty the loading device the cylinder should be held firmly and the piston should be moved toward the stent applicator. With this alternative embodiment, the cylinder does not need to be inserted so far into the stent applicator because the actual insertion of the stent into the applicator occurs by the piston's inserting action.

The fulfillment of this objective, where the device is concerned, is characterized by a cylinder-piston arrangement with a cylinder that is slit in the longitudinal direction at least partially and with a piston mounted so that it can slide inside the cylinder.

With the inventive device it is now possible for the first time to move even wire stents into a stent applicator, almost completely without manual handling and thus without risk of injury for the user.

According to a preferred embodiment of the invention it is proposed that the cylinder should be configured as a tube slit in the longitudinal direction with a handle mounted on the proximal end, so that the slit reaches as far as the distal end of the cylinder tube. This type of configuration of the cylinder allows for ease of handling the device as well as simple and inexpensive production.

According to the invention, the handle for operating the cylinder is advantageously configured as a disc mounted on the proximal end of the cylinder tube, so that the disc-shaped configuration of the handle ensures good and firm gripping of the handle.

It is further proposed with the invention that the piston should be configured as a plunger that is rod-shaped and mounted essentially form-locked in the cylinder tube and so that it can slide in the longitudinal direction of the cylinder tube, with a handle positioned on the proximal end. While the cylinder tube serves as a recess for inserting the stent, the piston configured as a rod-shaped plunger acts as an ejector tool to empty the cylinder tube and move the stent into the applicator.

According to a practical embodiment of the invention, it is proposed that the handle should be configured as a disc that is mounted on the cylinder tube and is connected by a stud with the piston in the area of the slit configured in the cylinder tube.

Finally, it is proposed with the invention that the stents should preferably be configured as wire-mesh stents. The inventive loading device is especially suited for every type of wire stent because it leads to no manual contact with the sharp stent ends that present a high risk of injury.

Further characteristics and advantages of the invention can be seen from the appended illustrations, in which one embodiment of an inventive device for loading a stent applicator is shown in a strictly exemplary way, with restricting the invention to this embodiment.

### BRIEF DESCRIPTION OF THE DRAWINGS

FIG. 1 shows a perspective side view of an inventive device for loading a stent applicator in disassembled state.

FIG. 2 shows an overhead view of the device according to FIG. 1 but showing the device assembled and with the stent inserted.

FIG. 3a shows a side view of a stent in withdrawn position.

FIG. 3b shows a side view of the stent according to FIG. 3a reduced in diameter by exerting a lateral force.

### DETAILED DESCRIPTION OF THE INVENTION

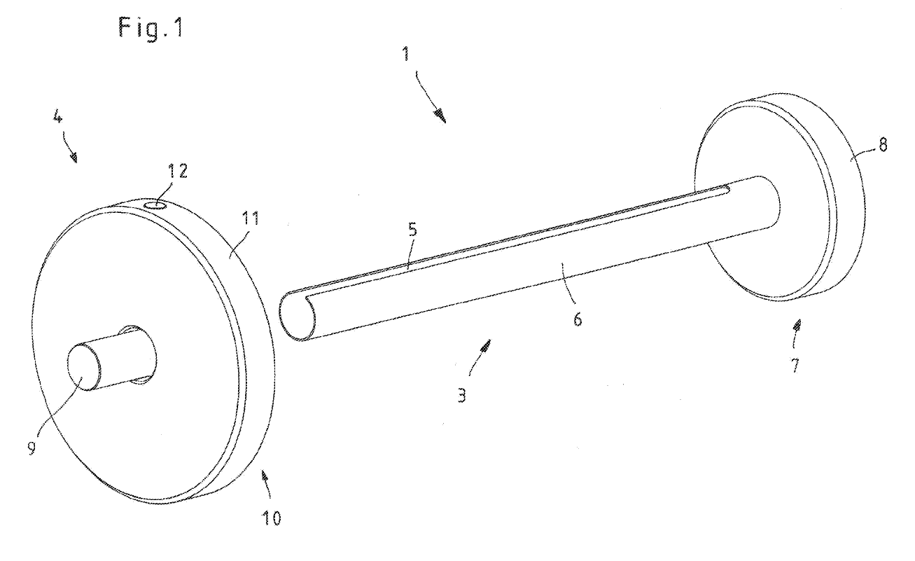

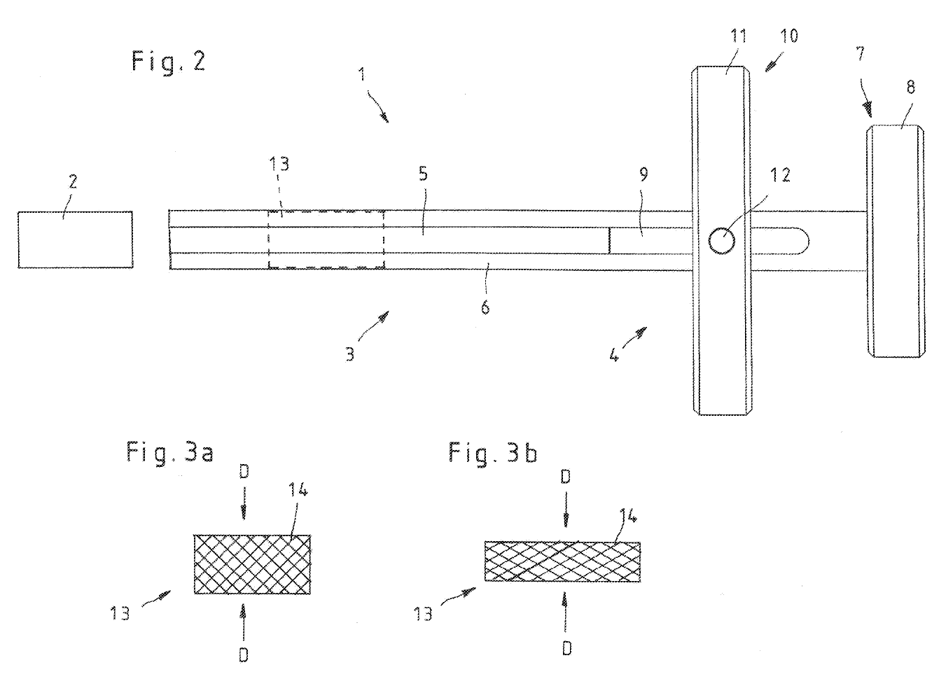

The loading device 1 for loading a stent applicator 2, shown in FIGS. 1 and 2, consists essentially of a cylinder-piston arrangement that includes a cylinder 3 and a piston 4.

As can be seen from FIGS. 1 and 2, the cylinder 3 is configured as a tube 6 that includes at least partially a longitudinal slit 5, so that the longitudinal slit 5 reaches to the distal end of the cylinder tube 6. Positioned on the proximal end of the cylinder tube 6 is a handle 7 which is configured in the illustrated embodiment as a disc 8 mounted on the proximal end of the cylinder tube 6.

The piston 4 is configured as a rod-shaped plunger 9 that is essentially form-lock mounted in assembled state in the loading device 1 and can be slid in the cylinder tube 6 in the longitudinal direction of the cylinder tube 6. Positioned at the proximal end of the piston is a handle 10 which is configured in the illustrated embodiment as a disc 11 that is mounted on the cylinder tube 6 and is connected by a stud 12 with the plunger 9 in the area of the slit 5 configured in the cylinder tube 6. As can be seen from FIG. 2, the stud 12 serves simultaneously as a rotation-proof guide in pushing the plunger 9 along the slit 5 of the cylinder tube 6.

The loading of a stent applicator 2 with a stent 13 by means of the aforementioned loading device 1 proceeds as follows.

Starting from the loading device 1 in the disassembled state shown in FIG. 1, the cylinder-piston arrangement is first completed by inserting the plunger 9 of the piston 4 into the cylinder tube 6. The stud 12 that connects the plunger 9 with the handle 10 now facilitates the tilt-proof insertion of the plunger 9 into the cylinder tube 6.

With the loading device 1 in the assembled, readied condition shown in FIG. 2, the cylinder tube 6 surrounds the plunger 9 coaxially all the way to the area of the slit 5 and essentially in form-locking manner.

The stent 13 is inserted into the cylinder 3 of the loading device 1 by the slit 5 configured in the cylinder tube 6. For this purpose the stent 13, as shown in FIGS. 3a and 3b, is reduced in diameter by exerting a lateral pressure D to the point where the stent 13 can be inserted into the cylinder tube 6 through the slit 5. FIG. 2 shows an overhead view of the loading device 1 with a stent 13 positioned in the cylinder tube 6.

Precisely with wire-mesh stents, the exertion of the lateral pressure D on the stent 13, because of the mutually slidable wires 14 of the wire mesh, causes a reduction of the outer diameter with simultaneous extending of the stent 13, as shown in exemplary enlargement in FIGS. 3a and 3b, where FIG. 3a shows the stent 13 in removed state and FIG. 3b shows the stent 13 reduced in diameter by exertion of the lateral pressure D but simultaneously extended in length.

Exertion of the lateral pressure D on the stent 13 is accomplished manually as a rule, but it is also possible to use a pressing device, for instance in the form of a forceps or a band, to exert the lateral pressure D on the stent 13. Such a band that can be used as a pressing device can be, for instance, commercially available cable ties.

Hereafter the cylinder tube 6 of the loading device 1 is inserted into the stent applicator 2 with the distal end forward until the stent 13 is completely inserted in the stent applicator 2.

To move the stent 13 into the stent applicator 2, the piston 4 is now pushed toward the stent applicator 2 until the distal end of the plunger 8 is contiguous with the proximal end of the stent 13 that is still situated in the cylinder tube 6. The actual emptying of the loading device 1 or loading of the stent applicator 2 occurs in the final step of the method. Here the piston 4 is fixed in place by the handle 10 in its position with respect to the stent applicator 2 and the cylinder 3 is pulled out of the stent applicator 2 by the handle 7.

This relative motion between the piston 3 and the cylinder 4 has the effect that the stent 13 is held in its position in the stent applicator 2 by the plunger 9 of the piston 4 when the cylinder 3 is drawn back, and after the complete drawing back of the cylinder 3 is placed completely and ready for the following surgical placement in the stent applicator 2.

As an alternative to the aforementioned manner of emptying the loading device 1 or loading the stent applicator 2, it is also possible to actively push the stent 13, which is positioned in the cylinder tube 6, by the plunger 9 of the piston 4 out of the cylinder tube 6.

With this embodiment of the loading method, the cylinder tube 6 is inserted only a short distance into the stent applicator 2 and then, while fixing the cylinder 3 in this position in the stent applicator 2, the piston 4 is pushed forward to the stent applicator 2 until the piston 9 has pressed the stent 13 completely out of the cylinder tube 6 and into the stent applicator 2.

The loading device 1 described above is particularly advantageous for loading a stent applicator 2 with wire-mesh stents 13, because the actual insertion of the stent 13 into the stent applicator 2 occurs without manual action, avoiding any risk of injury for the user of the loading device 1 by the sharp, pointed ends of the wire-mesh stents 13.

1. A method for loading a stent applicator, characterized by these steps:
a) Exert a lateral pressure on the stent to reduce its diameter,
b) Insert the compressed stent into a loading device that includes a cylinder-piston arrangement,
c) At least partially insert the loading device into the stent applicator, and
d) Load the stent applicator with the stent by pushing the components of the loading device cylinder and piston relative to one another.

2. The method according to claim 1, wherein in step a) of the method the lateral pressure is exerted on the stent manually.

3. The method according to claim 1, wherein in step a) of the method the lateral pressure is exerted on the stent by means of a pressing device.

4. The method according to claim 1, wherein the stent in step b) of the method is inserted through a lateral slit in the loading device.

5. The method according to claim 1, wherein in step d) of the method, for emptying the loading device, the piston is held firmly and the cylinder is moved away from the stent applicator.

6. The method according to claim 1, wherein in step d) of the method, to empty the loading device, the cylinder is held firmly and the piston is moved toward the stent applicator.

7. A device for loading a stent applicator, characterized by a cylinder-piston arrangement with a cylinder that is slit at least partially in the longitudinal direction and a piston mounted so that it can slide within cylinder.

8. The device according to claim 7, wherein the cylinder is configured as a tube that is slit in the longitudinal direction with a handle positioned on the proximal end, so that the slit extends to the distal end of the cylinder tube.

9. The device according to claim 8, wherein the handle is configured as a disc mounted on the proximal end of the cylinder tube.

10. The device according to claim 7, wherein the piston is configured as a rod-shaped plunger that is mounted essentially form-locked in the cylinder and can be slid in the longitudinal direction of the cylinder with a handle positioned on the proximal end.

11. The device according to claim 10, wherein the handle is configured as a disc that is mounted on the cylinder tube and that is connected with the piston by a stud in the area of the slit configured in the cylinder tube.

12. The device according to claim 7, wherein the stents are preferably configured as wire-mesh stents.

13. A pressing device for exerting a lateral pressure on a stent for reducing its diameter, characterized by at least one pressure means that surrounds the stent at least partially.

14. The pressing device according to claim 13, wherein the at least one pressure means is configured as jaw members of a forceps-type tool.

15. The pressing device according to claim 13, wherein the at least one pressure means is configured as a flexible band that surrounds and compresses the stent.
